(19) United States
(12) Patent Application Publication
(10) Pub. No.: US 2024/0363023 A1
(43) Pub. Date: Oct. 31, 2024

(54) Multipurpose Spatiomotor Capture System for Both Non-Visual and/or Visual Training and Testing in the Blind and the Sighted

(71) Applicants: Lora T. Likova, Greenbrae, CA (US); Christopher W. Tyler, San Francisco, CA (US)

(72) Inventors: Lora T. Likova, Greenbrae, CA (US); Christopher W. Tyler, San Francisco, CA (US)

(21) Appl. No.: 18/141,335

(22) Filed: Apr. 28, 2023

(51) Int. Cl. G09B 21/00; G16H 20/70; G16H 40/67

(52) U.S. Cl. CPC G09B 21/007; G16H 20/70; G16H 40/67

(57) ABSTRACT

A multipurpose capture system for both the training and behavioral assessment of spatial cognition, such as spatial memory and spatial analysis, and motor control for both navigation and manual performance. In one embodiment the system consists of two touchscreen tablet computers that share a common third monitor 4, and may share additional interface devices if necessary (e.g., keyboard and mouse) via wireless connections A participant is positioned adjacent to the pair of tablets and being unsighted, cannot visually process the stimuli, or the exploration or drawing trajectories. The participant explores the non-visual image structure with hand or fingers, the non-visual image being provided by raised-line tactile images, and/or vibration, and/or sound. A variety of memory-guided drawing tasks are performed on a tablet, and the results are interpreted to provide feedback to the participant.

### CROSS-REFERENCE TO RELATED APPLICATIONS

This application claims the priority date benefit of U.S. Provisional Application 63/336,500 filed Apr. 29, 2022.

### FEDERALLY SPONSORED RESEARCH

This invention was developed in work supported by grant funding: NIH/NEI ROI EY024056 & NSF/SL-CN 1640914.

### SEQUENCE LISTING, ETC ON CD

Not applicable.

### BACKGROUND OF THE INVENTION

### Field of the Invention

This invention relates to the fields of training and behavioral assessment of spatial cognition, such as spatial memory, mental representations/maps, spatial analysis, and motor decision-making and control for both navigation and manual spatiomotor performance, among individuals who are blind, visually impaired, or blindfolded-sighted, and thus cannot process visual stimuli, and also sighted individuals wishing to enhance their spatial memory and cognition through memory-guided drawing. More particularly, it relates to a multipurpose spatiomotor capture system that may be used for rehabilitation in blindness and visual impairment, or enhancement of spatial memory and cognition in sighted individuals.

### Description of Related Art

For those who have lost vision, the eye-hand coordination normally available for the manipulation of objects for everyday activities is unavailable and has to be replaced by information from other senses. It becomes crucial to activate cross-modal brain plasticity mechanisms for functional compensation of the visual loss in order to develop robust non-visual mental representations of space and objects. Such non-visual ‘mental maps’ are needed to guide spatiomotor coordination, reasoning and decision-making. Our multidisciplinary approach to this problem^{1,3,10,11} overcomes the shortcomings of traditional rehabilitation training, which can be both tedious and expensive.

For this purpose, Likova has developed an effective rehabilitation tool, the Cognitive-Kinesthetic (C-K) training approach to bridge the gap to wide spectrum blind rehabilitation by employing an integral task (drawing) that can affect ‘at one stroke’ a wide vocabulary of core abilities that are building blocks for numerous everyday tasks. For optimal implementation of this form of training, we have developed a multifunctional tactile/kinesthetic stimulation delivery and spatiomotor recording system for the enhancement of spatial memory functions through non-visual stimulation and recording devices. Understanding the behavioral and neural adaptation mechanisms underlying the rehabilitation of vision loss will also meet the broader goal of providing for a well-informed learning approach to functional rehabilitation. Our research of the behavioral and neural adaptation mechanisms underlying the rehabilitation of vision loss^{1-5,8,10} also meets the broader goal of providing for a well-informed learning approach to functional rehabilitation, such as our Cognitive-Kinesthetic (C-K) Training Method^{1-5,10},

The novel Cognitive-Kinesthetic (C-K) Training Method implemented in this system^{1-5,10} is based on the spatiomotor task of drawing, because drawing—from artistic to technical—is a ‘real-life’ task that uniquely incorporates diverse aspects of perceptual, cognitive and motor skills, thus activating the full ‘perception-cognition-action loop’³. Drawing engages a wide range of spatial manipulation abilities (e.g., spatio-constructional decisions, coordinate transformations, geometric understanding and visualization), together with diverse mental representations of space, conceptual knowledge, motor planning and control mechanisms, working and long-term memory, attentional mechanisms, as well as empathy, emotions and forms of embodied cognition^{1,3}. The Likova Cognitive-Kinesthetic Training Method makes it possible to use drawing as a ‘vehicle’ for both training and studying training-based cross-modal plasticity throughout the whole brain, including visual areas activated by non-visual tasks.

The innovative philosophy of this methodology is to develop an array of spatial cognition, cognitive mapping and enhanced spatial memory capabilities to provide those with compromised vision to develop—in a fast and enjoyable manner—precise and robust cognitive maps of desired spatial structures independently of a sighted helper; furthermore, to develop the ability to use these mental representations for precise motor planning and execution. See U.S. Pat. Nos. 10,307,087 and 10,722,150.

### BRIEF DESCRIPTION OF THE DRAWINGS

FIG. 1 is a functional layout of the in-person system for non-visual and visual training and testing of the present invention.

FIG. 2 is a functional block diagram of the remotely operated system for non-visual and visual training and testing of the present invention.

### DESCRIPTION OF PREFERRED EMBODIMENTS

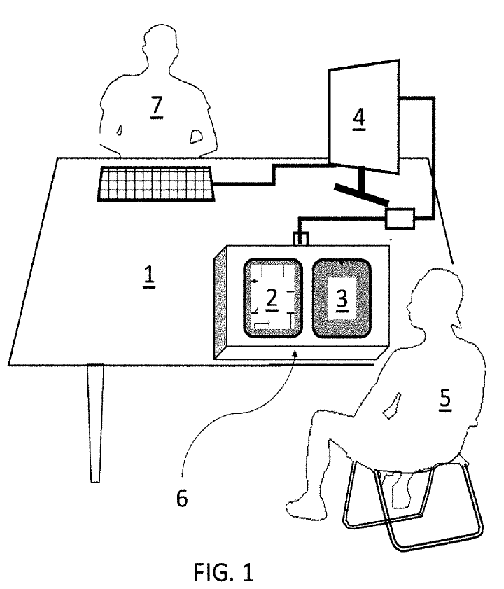

The multipurpose capture system of the invention serves a whole range of functions in both the training and behavioral assessment of spatial cognition, such as spatial memory and spatial analysis, and motor control for both navigation and manual performance. In more detail, in one embodiment the multipurpose spatiomotor capture system consists of a table 1 supporting two electronic devices, such as touchscreen tablet computers 2 and 3 (e.g., Microsoft Surface Pro tablet computers) that share a common third monitor 4, and may also share additional interface devices if necessary (e.g., keyboard and mouse) via wireless connections (FIG. 1). The participant 5 sits before the pair of tablets and being blind, visually impaired or blindfolded-sighted, cannot visually process the stimuli, or the exploration or drawing trajectories. Instead, they explore the non-visual image structure with their hand or fingers. The non-visual image structure can be provided by i) raised-line tactile images, ii) vibration, iii) sound (see the audio-haptic method in U.S. Pat. Nos. 10,307,087 and 10,722,150), or iv) by a combination of some or all of these presentation approaches. The pixel coordinates of the finger touch and button status on the touchscreen surfaces are recorded at high frequency (e.g., at 400 Hz) to appear virtually instantaneously to the test subject and operator. In the embodiment shown in FIG. 1, the left touchscreen 2 is used for the ‘Explore and Memorize’ tasks, and a sheet with a raised-line tactile image is placed over the touchscreen 2 and the time course of exploration of the tactile content is recorded by the tablet.

After tactile exploration of the raised-line content on tablet 2, a variety of memory-guided drawing tasks are performed on tablet 3 with data similarly recorded. Observational-drawing tasks can be included as well. Observational drawing is when the test subject explores by hand the tactile image with one hand such as on tablet 2, while drawing it with the other hand on tablet 3. Non-visual presentation of the spatial images to be haptically explored on tablet 2 can be implemented by the audio-haptic method from U.S. Pat. Nos. 10,307,087 and 10,722,150, where the tactile raised lines are replaced by audio signals that guide their haptic exploration along the image structure, instead of the tactile sensation of raised lines or surfaces.

The position of the two tablets can also be switched to accommodate participants of different handedness. For the integrated system 6, the tablet computers sit in recessed cut-outs in an adjustable-angle drawing board which is secured to a table. The recessed rectangles provide a tactile cue for the boundaries of the touchscreens, as well as holding the raised line tactile sheets in place on the left-hand side. The operator 7 can sit next to the participant in order to guide the hand and provide direct feedback during the initial training. Alternatively, the operator can sit on the opposite side of the table from the participant and control both tablets via the third computer 4 in order to manage the system without direct contact with the participant.

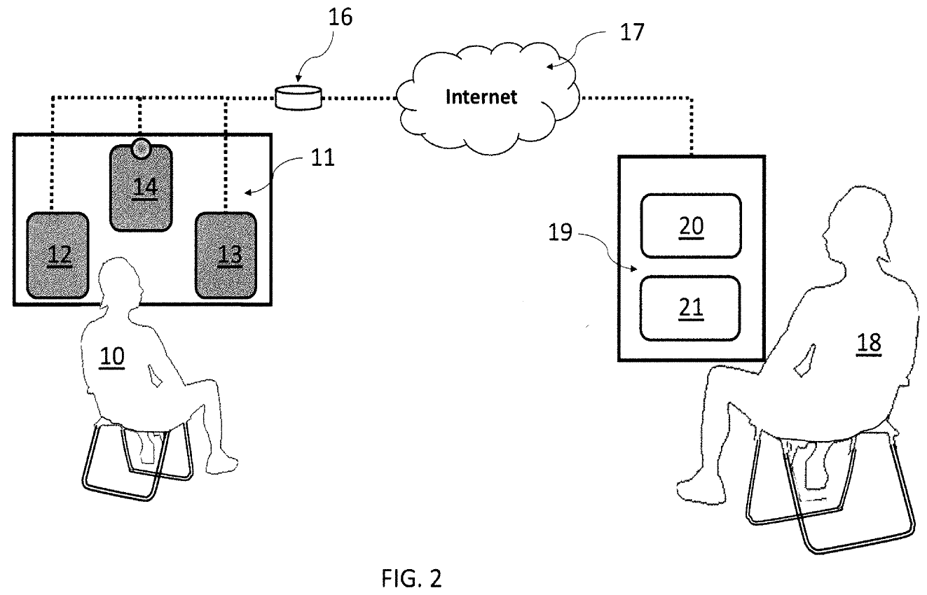

For the remote version of the system depicted in FIG. 2, the participant 10 sits before an integrated computer system 11 consisting of three tablet computers 12, 13, 14. The tablet computer 12 is used for haptic or visual exploration of tactually, audio-haptically or visually presented stimuli, which are thus memorized and then drawn on tablet computer 13. The third tablet computer 14 is mounted with a wide field camera affording a view of the participant's hands and face. The signals from these three computers are transmitted via a wifi router 16 to the cloud internet 17 and thence to a remote operator 18 who views the signals on a computer 19 running both a remote control app 20 that controls the operations on the three tablet computers 12-14 and a video conferencing app 21 that allows the operator 18 to view the hand movements and facial expression of the participant 10 and for them to communicate verbally and visually.

Software developed for the invention in Matlab provides a graphical user interface allowing for selection of which particular tactile stimuli are being presented as well as for initiation of data acquisition of the participant's drawing movements. Data acquisition is started and stopped by the operator with a keystroke on the keyboard. While data are being acquired, an image of the stimulus being either explored or drawn from memory is shown on the operator's display. Overlaid on the stimulus image, the exploration or drawing data are visualized as they accumulate to give the operator real-time feedback for how well the participant is performing. The exploration or memory-guided drawing data are stored for offline analyses of speed, accuracy and other features of hand-motion trajectories such as exploration strategies and speed-accuracy trade-offs at different stages of the learning process.

### Advantages

While the main system description is oriented toward non-sighted application for those without sight or with low vision, the multipurpose capture system is advantageous for use in a wide variety of other applications and populations, such as in the sighted and in populations with memory deficiencies.

### Haptic Rehabilitation Training

A key application of the system is for the haptic training of pictorial recognition and non-visual spatial memory for graphic materials such as those encountered in science, technology, engineering and mathematical (STEM) learning contexts or navigation guiding maps. Images and diagrams converted to raised-line tactile images may be explored and understood by haptic exploration with one or more fingers. In many cases, this is a novel medium for blind users familiar with exploration of objects as three-dimensional structures, as they learn to appreciate how objects such as faces can be represented in two dimensions. Once the images are encoded, the multipurpose capture system provides for the iterative exploration and drawing procedure, based on the capture of the drawing of the image on the second tablet by the hand that was not involved in the exploration. The switch between hands is an important advantage of the two-tablet system because it enforces the development of an accurate spatial memory of the image structure rather than simply relying on muscle memory, as would be possible if done with the same hand.

### Supervised/Unsupervised Learning Functionality

Another advantage of the system is its use in either supervised or unsupervised learning modes. In the supervised mode, the operator guides the participant through the procedures, based on the principles of the Likova Cognitive-Kinesthetic Training methodology. Briefly, these involve activating the perceptual-cognitive-motor loop through an elaborate supervised training process (see also U.S. Pat. Nos. 10,307,087 and 10,722,150). In the supervised mode, the operator can guide the participant in either local or remote mode. In the local mode, the operator sits adjacent to the participant through the procedure, while in the remote module the operator observes and guides the participant remotely. In the unsupervised mode, the participant works directly with the system after initial instructions on its operation. Automated feedback in this mode is provided by a computer algorithm, such as the Computerized Recognizability Index (CRI), developed in our lab, which compares the drawn configuration with the explored line-image and provides a measure of its accuracy (after affine transformations are taken into account). Its criteria are slightly different from human assessments of the accuracy, but it provides a quantitative index of improvement to guide the participant towards enhanced memory and motor-execution accuracy.

### Remote Operation

It is often necessary to conduct such training under remote operational conditions, e.g., as under requirements such as of the COVID-19 pandemic, which made it impossible to work with human subjects in the lab. There are many other situations that also greatly benefit from a remote operation, such as when participants experience difficulties to travel every day during the 5-day Cognitive-Kinesthetic Training. A remote version of the system has therefore been developed to allow the training station to be installed in the participant's domicile while the operator has remote access for the supervised training via an internet connection, as diagrammed in FIG. 2. The data are saved on computers 12 and 13 for transmission and storage on the host computer 19 or a server. This configuration requires a third tablet computer 14 with a camera to allow the operator to communicate verbally with the participant and observe the participant's movements and facial expressions to be able to provide the optimal feedback for effective training.

The information about the participant's activities from all three sources is transferred by internet connections to the host computer 19 or a server for storage and analysis. The control of the three-tablet system at the participant's station is managed by a remote control application on the host computer, and the two-way interchange of verbal and visual information between the operator and the participant is provided by a virtual communication application, such as Zoom software.

The system in this embodiment has been constructed on an adjustable table-top drawing table housing side-by-side Microsoft Surface Pro tablet computers, although other computer devices and supporting structures are also possible. The computers are conveniently programmable for the presentation and recording of the navigation learning and drawing of the learned trajectories for subsequent trajectory analysis. The operator's control is facilitated by the integration of a third electronic device, such as a tablet computer into the system for real-time monitoring of the output trajectories, as shown in FIG. 2. This greatly widens the scope of participant access and convenience for both participants and operators, and in particular in times such as pandemics this is a breakthrough feature.

### Visual Training of Spatial Memory

A further application of the two-tablet system is for visually-guided training in sighted users in the same manner as the haptic/motor training, implementing a visual version of the Likova Cognitive-Kinesthetic Drawing Training¹⁻¹⁰. In this case, the procedure can all be carried out on a single electronic device, such as tablet computer or a smart phone (with a second one for the remote monitoring and training interchanges if being conducted remotely). STEM or art images or maps can be presented visually on the screen and explored visually rather than tactilely, and can then disappear to show a blank screen for the memory-guided drawing phases. The drawing can be done either entirely from memory with no visual feedback, or in the manner of a conventional drawing in which the image appears progressively as it is being drawn, providing continuous feedback of the drawing result to be compared with the internal memory of what has to be drawn. This conventional drawing approach should be less effective at training accurate spatial memory than the approach with no immediate feedback, in which the entire drawing trajectory is guided from memory according to principles of the Likova Cognitive-Kinesthetic Training¹⁻¹⁰, then comparing the finished result with the original to provide global feedback about its success. In this way, the vividness and practical applicability of spatial memory can be maximally enhanced in only a short period of training. Training regimens other than the Likova C-K Training may also be implemented using this invention.

### CONCLUSION

Our Multipurpose Spatiomotor Capture System for Both Non-Visual and/or Visual Training and Testing in the Blind and the Sighted is both a powerful novel conceptualization and a tool for both research and applied purposes, such as neuro-rehabilitation, or the enhancement of spatial cognition, learning and memory in children. It makes it possible to implement advanced training procedures, such as the unique Cognitive-Kinesthetic drawing and spatial memory training; and, moreover, to implement it both in-person and in a remote mode of operation in a wide range of populations—from the totally blind to the fully sighted.

### REFERENCES

1. Likova L T Drawing enhances cross-modal memory plasticity in the human brain: A case study in a totally blind adult. Frontiers in Human Neuroscience 2012, 6:44.
2. Likova L T The spatiotopic ‘visual’ cortex of the blind. Human Vision and Electronic Imaging XVII, 2012, 8291-10L.
3. Likova L T A cross-modal perspective on the relationships between imagery and working memory. Front. Psychology 2013, 3:561.
4. Likova L T Learning-based cross-modal plasticity in the human brain: insights from visual deprivation fMRI. Advanced Brain Neuroimaging Topics in Health and Disease-Methods and Applications, 2014, 327-358.
5. Likova L T Temporal evolution of brain reorganization under cross-modal training: Insights into the functional architecture of encoding and retrieval network. Human Vision and Electronic Imaging XX, 2015, 9394: 939417-33.
6. Likova L T Granger Causality analysis reveals the role of the hippocampal complex in the memory functions of primary visual cortex. European Conference on Visual Perception Abstracts. Perception 2017
7. Likova L T Addressing long-standing controversies in conceptual knowledge representation in the temporal pole: A cross-modal paradigm. Human Vision and Electronic Imaging, 2017, 268-272(5).

1. A system for training spatial cognition, spatial memory and spatiomotor performance comprising a device for presenting a tactile spatial image to be explored by hand and a second device that encodes the trajectory generated by drawing said image from memory.

2. The system of claim 1 wherein said tactile image is presented by either a raised line image in a physical medium or a by a tactile representation on the screen of a tablet computer conveyed by a means such as auditory or vibratory coding based on the position of a finger or stylus relative to said tactile image line on said screen.

3. The system of claim 1 wherein the degree of correspondence between said drawing and said spatial image is calculated computationally and reported to said participant by said system.

4. The system of claim 1 wherein the correspondence between said drawing and said spatial image is evaluated by a human operator and strategies for improvement are reported verbally to said participant by said system.

5. The system of claim 1 wherein said devices for presenting said image and encoding said drawn trajectory are tablet computers and a third device provides for two-way remote communication between said system and a remote computer through which an operator remotely controls said system to select each of several images for drawing and managing the provision of said reporting of said participant's performance for said purpose of training of spatial cognition, spatial memory and spatiomotor performance.

6. The system of claim 1 wherein said spatial image is presented visually.

7. The system of claim 6 wherein the degree of correspondence of said drawing with said spatial image is calculated computationally and reported to said participant by said system.

8. The system of claim 6 wherein the correspondence of said drawing with said spatial image is evaluated by a human operator and strategies for improvement are reported verbally to said participant.

9. The system of claim 6 wherein said devices for presenting said image and encoding said drawn trajectory are tablet computers and further including a third device providing for two-way remote communication between said system and a remote computer through which an operator remotely controls said system to select each of several images for drawing and managing the provision of said reporting of said participant's performance for said purpose of training of spatial cognition, spatial memory and spatiomotor performance.

10. A process for training spatial cognition, spatial memory and spatiomotor performance comprising means for presenting a tactile spatial image to be explored by hand and means for encoding the trajectory generated by drawing said image from memory.

11. The process of claim 10 wherein said tactile image is presented by either a raised line image in a physical medium or a by a tactile representation on the screen of a tablet computer conveyed by a means such as auditory or vibratory coding based on the position of a finger or stylus relative to said tactile image line on said screen.

12. The process of claim 11 wherein the degree of correspondence between said drawing and said spatial image is calculated computationally and reported to said participant through said process.

13. The process of claim 11 wherein the correspondence between said drawing and said spatial image is evaluated by a human operator and strategies for improvement are reported verbally to said participant through said process.

14. The process of claim 11 wherein said means for presenting said image and encoding said drawn trajectory comprise a pair of tablet computers, and a third device is connected for two-way remote communication between said process and a remote computer through which an operator remotely controls said process to select each of several images for drawing and managing the provision of said reporting of said participant's performance for said purpose of training of spatial cognition, spatial memory and spatiomotor performance.

15. The process of claim 11 wherein said spatial image is presented visually.

16. The process of claim 15 where the degree of correspondence of said drawing with said spatial image is calculated computationally and reported to said participant by said process.

17. The process of claim 15 wherein the correspondence of said drawing with said spatial image is evaluated by a human operator and strategies for improvement are reported verbally to said participant.

18. The process of claim 15 wherein said means for presenting said image and encoding said drawn trajectory are tablet computers and a third process provides for two-way remote communication between said participant and a remote computer through which an operator remotely controls said process to select each of several images for drawing and managing the provision of said reporting of said participant's performance for said purpose of training of spatial cognition, spatial memory and spatiomotor performance.
